SEQUENCE LISTING (10 sequences)

SEQ ID NO: 1 - DNA, 20 bp, Artificial Sequence
  human Nestin forward primer
ctctgacctgtcagaagaat

SEQ ID NO: 2 - DNA, 20 bp, Artificial Sequence
  human Nestin reverse primer
gacgctgacacttacagaat

SEQ ID NO: 3 - DNA, 20 bp, Artificial Sequence
  human b-tubulin III(TuJ1) forward primer
atgagggagatcgtgcacat

SEQ ID NO: 4 - DNA, 18 bp, Artificial Sequence
  human b-tubulin III(TuJ1) reverse primer
cccctgagcggacactgt

SEQ ID NO: 5 - DNA, 21 bp, Artificial Sequence
  human NF-M forward primer
tgggaaatggctcgtcatttg

SEQ ID NO: 6 - DNA, 21 bp, Artificial Sequence
  human NF-M reverse primer
cttcatggaaacggccaattc

SEQ ID NO: 7 - DNA, 21 bp, Artificial Sequence
  human NF-L forward primer
tcctactacaccagccatgtc

SEQ ID NO: 8 - DNA, 21 bp, Artificial Sequence
  human NF-L reverse primer
tccccagcaccttcaactttc

SEQ ID NO: 9 - DNA, 22 bp, Artificial Sequence
  human beta-actin forward primer
ccacgaaactaccttcaactcc

SEQ ID NO: 10 - DNA, 25 bp, Artificial Sequence
  human beta-actin reverse primer
tcatactcctgctgcttgctgatcc

(19) United States
(12) Patent Application Publication
(10) Pub. No.: US 2008/0176328 A1
(43) Pub. Date: Jul. 24, 2008

(54) METHOD OF INDUCING DIFFERENTIATION OF MESENCHYMAL STEM CELLS INTO NEURONS

(75) Inventors: Mi-Sook Chang, Seoul (KR); Jung Sun Cho, Seoul (KR)

Correspondence Address: JHK LAW, P.O. BOX 1078, LA CANADA, CA, 91012-1078, US

(73) Assignee: Seoul National University Industry Foundation, Seoul (KR)

(21) Appl. No.: 11/625,252

(22) Filed: Jan. 19, 2007

(51) Int. Cl. C12N 5/02

(52) U.S. Cl. 435/377; 435/404

(57) ABSTRACT

The present invention relates to a method for inducing differentiation of bone marrow-derived mesenchymal stem cells into mature neurons by culturing them in an optimal medium supplemented with necessary composition. According to the pre-induction method of the invention and a method for inducing differentiation of mesenchymal stem cells into neurons by culturing them in neuronal induction media (NIM) containing butyl hydroxyanisole, forskolin and VPA, mesenchymal stem cells can be effectively differentiated into neurons or motor neurons, which thereby can be effectively used as a therapeutic agent for cell therapy for neurodegenerative diseases.

### TECHNICAL FIELD

The present invention relates to a method for inducing differentiation of bone marrow-derived mesenchymal stem cells into mature neurons by culturing them in an optimum medium supplemented with necessary composition.

### BACKGROUND ART

Stem cells are the cells of the pre-differentiation stage before being differentiated into each tissue forming cell, indicating that they have self-renewal capacity with unlimited proliferation potential before being differentiated and at the same time have pluripotency with potential for differentiation into various tissue cells by a specific stimulus. That is, even after repeated culture, self-renewal capacity does not decrease, and stem cells can be differentiated into various types of cells.

Stem cells are largely divided into embryonic stem cells (ES cells) and adult stem cells according to the differentiation potential. After a sperm meets an ovum, they are fertilized and developed to form a blastocyst. Embryonic stem cells are isolated from inner cell mass (ICM), which is supposed to be developed into a fetus, in the very early stage blastocyst before the fertilized egg is implanted in the endometrium. These embryonic stem cells are pluripotent cells that are able to be differentiated into every tissue generated from 3 embryonic germ layers (endoderm, ectoderm and mesoderm).

In the meantime, adult stem cells are organ specific stem cells that are isolated from an adult whose development has been completed or placenta in which organ forming stage is actively undergoing. Potency of those adult stem cells is pluripotent, which means the potency is generally limited to tissue forming cells. Adult stem cells remain in organs of even grown-up and thus play a role in supplementing normal or pathological cell loss. The most representative adult stem cells are hematopoietic stem cells in bone marrow and mesenchymal stem cells to be differentiated into connective tissue cells except blood cells. Hematopoietic stem cells are differentiated into various blood cells such as erythrocytes and leucocytes and mesenchymal stem cells are differentiated into osteoblasts, chondroblasts, adipocytes and myoblasts.

Recently human embryonic stem cells were successfully isolated and the clinical application thereof has been a major concern. The best interest of stem cell application is the use as a perfect cell supplier for cell replacement therapy. Neurodegenerative diseases such as Parkinson's disease and Alzheimer's disease, quadriplegia caused by spinal cord injury, leukemia, stroke, juvenile diabetes, myocardial infarction and liver cirrhosis are caused by the destruction of cells forming tissue and permanent functional disorder. To supplement cells to make up the lack of cells caused by cell destruction or malfunction, cell replacement therapy has been proposed.

Even though cell replacement therapy has been confirmed to have astonishing effect, it still has limitation for clinical application. The conventional method to supply cells is that fully differentiated cells are isolated from a donor and then transplanted to a patient. But it is very difficult to obtain cells enough for a patient. To solve the problem of short cell supply, both embryonic stem cells and adult stem cells can be isolated, proliferated and differentiated in vitro into a specific cell for cell replacement therapy.

However, due to their excellent self-renewal capacity, embryonic stem cells might induce teratoma and the proliferation of undifferentiated cells when they are transplanted in a living body for cell therapy. Since the efficiency in differentiation into specific target cells of embryonic stem cells is low, it might cause side effects by the cell blend with other non-targeted differentiated cells when they are transplanted in a patient. Therefore, more elaborate method of inducing differentiation is required for safer clinical application of embryonic stem cells.

In the meantime, cell replacement therapy using adult stem cells has also problems that cell proliferation is reduced under long-term culture; and/or differentiation potency might be modified so that differentiation into unwanted cells occurs. Neurodegenerative disease such as Parkinson's disease can be treated by neuron transplantation. But, it is very difficult to obtain neural stem cells directly from a patient. Thus, neural stem cells isolated from the fetal brain have been proliferated and differentiated into neurons in vitro for treatment. However, to treat one patient, generally two fetus brains are required, which causes ethical issues in addition to the shortage in supply. Moreover, most neural stem cells are differentiated into astrocytes in vitro rather than into neurons, and they can induce immune rejection.

If it is possible to differentiate bone marrow-derived mesenchymal stem cells into neurons, it will solve the problems of a short cell supply and immune rejection since autologous bone marrow is used. It has been a common belief so far that a kind of stem cells is differentiated only into a specific tissue cell belonging to the same lineage. Mesenchymal stem cells are able to form in vitro colonies in the presence of various growth factors such as platelet-derived growth factor, basic fibroblast growth factor, TGF-β (transforming growth factor-β) or EGF (Kuznetsov et al., Br. J. Haematol. 97:561, 1997; van den Bos C et al., Human Cell 10:45, 1997). Approximately ⅓ of early adherent cells have pluripotency, so that they can be differentiated into connective tissue cells such as osteoblasts, chondroblasts and adipocytes (Pittenger MF et al., Science 284:143, 1999). In addition, Ferrari et al reported previously that bone marrow is the source of myogenic precursor cells involved in the formation of new muscles (Ferrari G et al., Science 279:1528, 1998).

Recent reports say that mesenchymal stem cells used to be known to be differentiated only into connective tissues are differentiated into nerve cells, too. For example, Sanchez-Ramos et al reported that when mesenchymal stem cells were cultured in the presence of retinoic acid and BNDF (brain-derived neurotrophic factor), the cells were differentiated into neurons and astrocytes (Sanchez-Ramos et al., Exp. Neurology 164:247-256, 2000). In the meantime, Dale Woodbury et al reported that bone marrow-derived mesenchymal stem cells could be differentiated into neurons when they were cultured in the presence of antioxidants such as β-mercaptoethanol or DMSO (dimethyl sulfoxide) (Dale Woodbury et al., J. Neuro. Res. 61:364-370, 2000).

However, induction of differentiation of mesenchymal stem cells into neurons is still limited. First, a growth factor should bind to a specific growth factor receptor expressed endogenously in the cell for intracellular signal transmission. But, there has been no report about the expression of such growth factor receptor in mesenchymal stem cells, yet. Unless the expression of such a growth factor receptor is clearly detected, the concentration of a growth factor to activate a receptor cannot be determined. If a growth factor does not bind to a receptor under natural physiological environment (37° C.), it will be hydrolyzed very fast by various enzymes. Thus, the activity of such growth factor will vary in a medium. Therefore, many scientists have tried to induce differentiation of mesenchymal stem cells into neurons by using an induction medium containing antioxidants such as DMSO and BHA instead of a growth factor. However, the results were not satisfactory. Particularly, the differentiation of mesenchymal stem cells into neurons could be possible using DMEM supplemented with various compounds (Bertani N et al., J Cell Sci, 118, 3925-3936, 2005; Woodbury D et al., J Neurosci Res, 69, 908-917; Guillermo M-E et al., 21, 437-448, 2003), but reproducibility of the result was very low and the marker of differentiated neurons could not be detected (Bertani N, J Cell Sci, 118, 3925-3936). Zhao et al tried to induce stable differentiation of mesenchymal stem cells into neurons by adopting two-phase induction method (Zhao et al., Exp Neurol, 190, 396-406, 2004). Compared with the earlier trials, this attempt resulted in better differentiation, but the differentiation induction time was extended to 24 hours and the marker expression was not enough to confirm the differentiation of mesenchymal stem cells into mature neurons.

Therefore, the present inventors introduced two-phase pre-induction system and tried to optimize the condition of the neuronal induction medium. The present inventors also finally completed this invention by confirming the expression of a marker for differentiated mature neurons and reproducible differentiation of mesenchymal stem cells into neurons.

### DISCLOSURE

### Technical Problem

It is an object of the present invention to provide neurons useful for cell therapy by inducing reproducible differentiation of mesenchymal stem cells into neurons by using an optimized induction medium and a culture method.

### Technical Solution

To achieve the above object, the present invention provides a method for inducing differentiation of mesenchymal stem cells into neurons, comprising the following steps:

1) Performing pre-induction of mesenchymal stem cells twice; and

2) Inducing differentiation of the pre-differentiated mesenchymal stem cells of step 1) in a neuronal induction medium containing butylated hydroxyansiole (BHA), forskolin and valproic acid (VPA) for 2˜8 hours.

The present invention also provides a neuronal induction medium containing butylated hydroxyansiole (BHA), forskolin and valproic acid (VPA) to induce differentiation of pre-differentiated mesenchymal stem cells into neurons.

The mesenchymal stem cells hereinabove is preferably isolated from human bone marrow. Particularly, mononuclear cells are isolated from bone marrow, which are cultured for 1˜2 weeks. Then, differentiation-ready hematopoietic stem cells are all differentiated to generate mature blood cells and remaining stem cells are isolated, by which mesenchymal stem cells are obtained. In addition to the separation of mesenchymal stem cells from mononuclear cells isolated from bone marrow, the whole mononuclear cells containing mesenchymal stem cells can be cultured according to the method of the present invention to mass-produce neurons.

The method of inducing differentiation of mesenchymal stem cells into neurons is that pre-induction of step 1) is performed twice with increasing the concentration of β-mercaptoethanol and then differentiation is induced in a neuronal induction medium containing 100˜200 μM of BHA, 9˜11 μm of forskolin and 1.5˜2.5 mM of VPA. During the pre-induction, it is preferred to increase the concentration of β-mercaptoethanol for the second pre-induction up to 1.5˜2 fold, more preferably 2 fold, from the concentration for the first pre-induction. The second pre-induction time is preferably shortened to ¼˜⅛ of the first pre-induction time and ⅛ time is preferred. The neuronal induction medium preferably contains 200 μM of BHA, 10 μm of forskolin and 2 mM of VPA and induction time is preferably 2˜8 hours and more preferably 3˜5 hours (see FIG. 1 and FIG. 2). RT-PCR was performed to find that a neuronal marker expression was the highest in the group treated with NIM for 4 hours, whereas the immunofluorescent staining to detect a specific protein expression in each cell confirmed that NF-M expression was dominant in the group treated with NIM for 6 hours (see FIG. 5).

The butylated hydroxyanisole (BHA) is an organic compound comprising two isomers, 2-tert-butyl-4-hydroxyanisole and 3-tert-butyl-4-hydroxyanisole, and has the characteristics of an antioxidant. BHA is known to inhibit an intracellular signal pathway regulated by reactive oxygen intermediates, such as nuclear factor (NF)-kB activation (Sasada T et al., J. Clin. Invest., 97, 2268-2276, 1996). Thus, such an antioxidant activity leads to the increase in neuroprotection (Poeggeler B et al., J. Neurochem, 95, 962-973, 2005). Forskolin (7 beta-acetoxy-8, 13-epoxy-1 alpha, 6 beta, 9 alpha-trihydroxy-labd-14-ene-11-one) activates adenylcyclase and increases cyclic AMP (cAMP) to stimulate intracellular signaling pathway. cAMP is a crucial signal transmitter which is necessary for cell response and also plays an important role in activation of protein expression for cell survival and differentiation. Valproic acid (VPA) inhibits histone deacetylase (HDAC) activity directly and this inhibition suppresses cell growth, which leads to increases in the differentiation of tumor cells. VPA also activates signal transduction pathway such as extracellular signal-regulated kinase (ERK) pathway to increase cell survival (Hsieh J et al., PNAS, 101, 16659-16664, 2004). It is also reported that ERK is involved in the proliferation of differentiated cells (Joneson T et al., J Biol Chem, 273, 7743-7748, 1998; Christerson LB et al., Cell Motil Cytoskeleton, 43, 186-198, 1999; Stariha RL & Kim SU, Microsc Res Tech, 52, 680-688, 2001; Vaudry D et al., J Neurochem, 83, 1272-1284, 2002).

In general, the concentration of DMSO to fast-freeze cells is 10%. However, in the present invention, 2% DMSO was used only for 6 hours, so that cytotoxicity might not be induced. In the present invention, mesenchymal stem cells were isolated from human bone marrow and cultured in a medium designed to efficiently induce differentiation of mesenchymal stem cells into neurons in vitro, suggesting that there was no need to use this medium directly in human. That is, only cells differentiated in the medium can be isolated and injected into human body for therapy.

The present inventors re-conducted the earlier experiments to induce differentiation of mesenchymal stem cells into neurons (Zhao et al., Exp Neurol, 190, 396-406, 2004; Bertani N et al., J Cell Sci, 118, 3925-3936, 2005; Woodbury D et al., J Neurosci Res, 69, 908-917; Guillermo M-E et al., 21, 437-448, 2003). However, the result was not consistent with that of the earlier experiment. The efficiency of neuronal induction was very low, and marker expression was hardly detected, which means the differentiation of mesenchymal stem cells into neurons was difficult to be confirmed (see FIG. 7). In addition, the differentiation of mesenchymal stem cells into neurons was not enough and unsatisfactory (see FIG. 6). Thus, the present inventors developed a highly reproducible novel method for inducing differentiation of mesenchymal stem cells into neurons by eliminating unnecessary compounds for neuronal induction. Then, the expression of differentiated neuronal markers (neurofilament 150 kDa, NF-M; neurofilament 68 kDa, NF-L), including a motor neuron maker (Islet-1) was confirmed by RT-PCR (see FIG. 3). The present inventors further confirmed the expression of NF-M protein in the differentiated neurons by immunocytochemistry method (see FIG. 4). In addition, the inventors observed that the combination of the method of Zhao et al and the method of Bertani et al induced differentiation of mesenchymal stem cells into neurons more efficiently than the method of Zhao et al was used alone. Furthermore, expression of the marker for differentiated neuron was increased and this result was highly reproducible (see FIG. 9).

### DESCRIPTION OF DRAWINGS

The application of the preferred embodiments of the present invention is best understood with reference to the accompanying drawings, wherein:

FIG. 1 is a set of photographs illustrating the morphological changes over the times of treating NIM (neuronal induction media) to mesenchymal stem cells,

FIG. 2 is a set of photographs illustrating the expression levels of neuronal markers over the times of treating NIM (neuronal induction media) to mesenchymal stem cells,

FIG. 3 is a set of photographs illustrating the gene expressions of the NIM non-treated control group and the NIM-treated group,

FIG. 4 is a set of photographs illustrating the expressions of neuronal markers over NIM treating times (0, 3, 6, 9 and 24 hr), confirmed by immunofluorescence assay,

Red: nestin Green: NF-M Blue: DAPI

FIG. 5 is a set of photographs illustrating the expressions of neuronal markers over NIM treating times (2, 4, 6 and 8 hr), confirmed by immunofluorescence assay,

Red: NF-M Green: nestin

FIG. 6 is a set of microphotographs illustrating that differentiation of mesenchymal stem cells into neurons was induced by the method of Zhao et al, but the differentiation of mesenchymal stem cells into neurons was not successful,

FIG. 7 is a set of photographs illustrating the comparison of the gene expressions among the NIM-treated mesenchymal stem cell group, a mesenchymal stem cell group treated by the method of Zhao et al and the NIM non-treated group,

FIG. 8 is a set of microphotographs illustrating that differentiation of mesenchymal stem cells into neurons was comparatively clear when the method of Zhao et al was used along with the method of Bertani et al, compared with when the method of Zhao et al was used alone,

FIG. 9 is a set of photographs illustrating the comparison of gene expressions between the mesenchymal stem cell group treated by the combination of the method of Zhao et al and the method of Bertani et al and the non-treated group.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXPERIMENTAL EXAMPLE 1

### Recurrence of Differentiation of Mesenchymal Stem Cells into Neurons by the Method of the Present Invention

<1-1> Human Mesenchymal Stem Cell (hMSC) Culture

Poietics Normal Human Mesenchymal Stem Cells were purchased from Cambrex, USA. The above stem cells were subcultured two times in a MSC growth medium (MSCGM-500 ml of mesenchymal cell growth supplement, 10 ml of 200 mM L-glutamine, 0.5 ml of penicillin-streptomycin, Cambrex, USA) comprising a basic medium and a growth supplement, and then transferred into DMEM (Gibco, USA) supplemented with 10% FBS (Gibco, USA), 100 ng/ml penicillin and 100 U/ml streptomycin, followed by further culture in a 37° C., 5% CO₂ incubator. After three days of culture in the incubator, the medium was removed and the cells were washed with phosphate-buffered saline (PBS) to completely remove the remaining medium. Cells were detached with 0.1% trypsin/EDTA (Gibco, USA) and then diluted with a new medium at the ratio of 1:3, followed by subculture.

<1-2> Induction of Neuronal Differentiation of hMSC

Mesenchymal stem cells were subcultured in DMEM supplemented with 10% FBS and penicillin-streptomycin. To induce neuronal differentiation, the stem cells were cultured in a general medium containing 1 mM β-mercaptoethanol (Sigma, USA) for 24 hours and then the medium was replaced with another general medium containing 2 mM β-mercaptoethanol, followed by further culture for 3 hours (pre-induction). After the pre-induction, the medium was replaced once again with a neuronal induction medium (NIM) prepared by adding 2% DMSO (Sigma, USA), 200 μM butylated hydroxyanisole (Sigma, USA), 10 μM forskolin (Sigma, USA), 2 mM valproic acid (Sigma, USA) and 10 mM potassium chloride (Sigma, USA) to the DMEM supplemented with N2 supplements (Gibco, USA) instead of 10% FBS. After culture for a required times (0, 2, 4, and 8 hours), the changes of morphology were observed under the microscope.

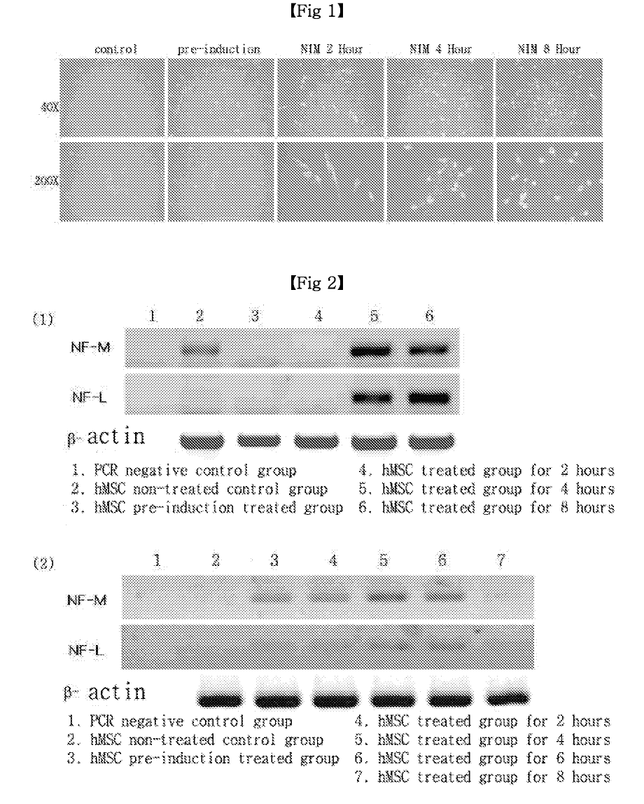

From the observation of morphology of the above experimental group was confirmed that the mesenchymal stem cells cultured in the NIM changed into neuron-like morphology (FIG. 1).

### <1-3> Detection of Neuronal Differentiation Markers by RT-PCR

RNA was extracted from both non-treated control mesenchymal stem cells and NIM-treated mesenchymal stem cells by using trizole (Invitrogen, USA). Reverse transcription (RT) was performed with 2 μg of the extracted RNA by using MMLV reverse transcriptase (MMLV RTase; Promega, USA). Particularly, RT was performed with 50 μl of volume by using 0.5 μg of oligo (dT) primer, 2.5 mm dNTPs, 5× MMLV buffer, RNase inhibitor and MMLV RTase. Then, PCR amplification was performed using a PCR machine (Bio-Rad, USA) as follows; predenaturation at 94° C. for 5 minutes, denaturation at 94° C. for 45 seconds, annealing at 55˜65° C. for 45 seconds, polymerization at 72° C. for 45 seconds, 35 cycles from denaturation to polymerization, and final extension at 74° C. for 7 minutes.

PCR was performed with the RT-product (3˜5 μl), using the primer set presented in Table 1. For the negative control, PCR was performed with water instead of the RT product. To increase accuracy of PCR result and equal distribution, a master mix composed of the primer set, 10× buffer, Taq polymerase, and 2.5 mM dNTPs was loaded in each reaction tube, to which RT product or water was added as a template, followed by PCR. PCR was performed as follows; predenaturation at 95° C. for 5 minutes, denaturation at 95° C. for 45 seconds, annealing at 65° C. for 45 seconds, polymerization at 72° C. for 45 seconds, 35 cycles from denaturation to polymerization, and final extension at 72° C. for 5 minutes, followed by cooling at 4° C. PCR product was electrophoresed on 1.5% agarose gel, and the band size was measured by using a transilluminator.

Table 1: PCR primer set

TABLE 1

| | | |
|---|---|---|
| Name | Sequence | Size |

| | | | |
|---|---|---|---|
| Human Nestin | Forward | CTCTGACCTGTCAGAAGAAT | 316 bp |
| | | (SEQ. ID. NO: 1) | |
| | Reverse | GACGCTGACACTTACAGAAT | |
| | | (SEQ. ID. NO: 2) | |
| Human b- | Forward | ATGAGGGAGATCGTGCACA | 267 bp |
| tubulin | | (SEQ. ID. NO: 3) | |
| III (TuJ1) | Reverse | CCCCTGAGCGGACACTGT | |
| | | (SEQ. ID. NO: 4) | |
| Human NF-M | Forward | TGGGAAATGGCTCGTCATTTG | 333 bp |
| | | (SEQ. ID. NO: 5) | |
| | Reverse | CTTCATGGAAACGGCCAATTC | |
| | | (SEQ. ID. NO: 6) | |
| Human NF-L | Forward | TCCTACTACACCAGCCATGTC | 285 bp |
| | | (SEQ. ID. NO: 7) | |
| | Reverse | TCCCCAGCACCTTCAACTTTC | |
| | | (SEQ. ID. NO: 8) | |
| Human beta- | Forward | CCACGAAACTACCTTCAACTCC | 285 bp |
| actin | | (SEQ. ID. NO: 9) | |
| | Reverse | TCATACTCCTGCTGCTTGCTGA | |
| | | TCC | |
| | | (SEQ. ID. NO: 10) | |

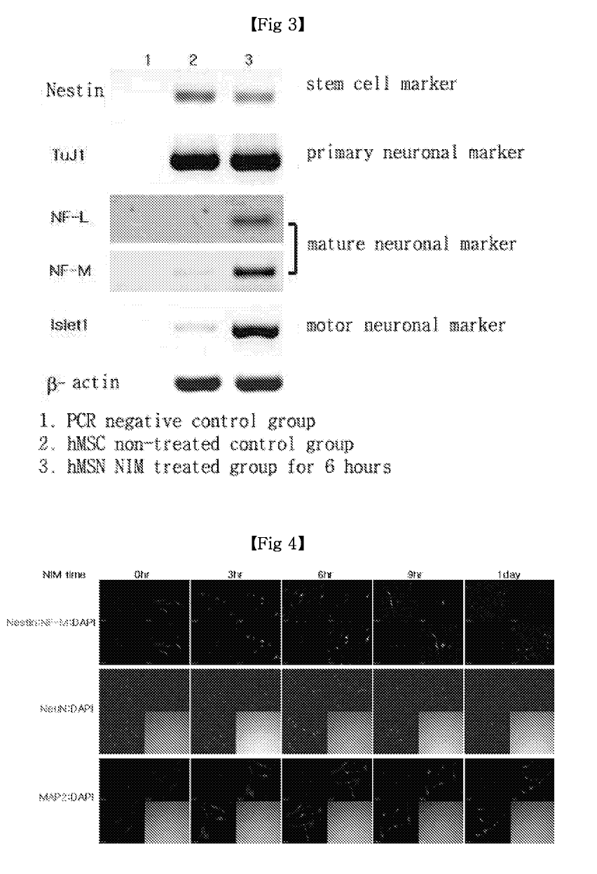

As a result, the expressions of NF-M and NF-L, mature neuronal markers, depended on the induction time by NIM in hMCS. Particularly, the expressions of NF-M and MF-L were hardly detected in the NIM-non-treated control group. After treating hMSC with NIM, the expressions of NF-M and NF-L were gradually increased over the times for 6 hours, which was the turning point to start decreasing of the levels (FIG. 2). More specifically, NIM treatment of hMSC for 4 hours highly induced differentiation of mesenchymal stem cells into neurons. Considering that mature neuronal marker expression was the highest, induction time was preferably determined to be 4 hours for efficient induction. Even though the primary neuronal marker expression of the NIM-treated group was similar to that of the non-treated group, the expressions of mature neuronal markers (NF-M and NF-L) and motor neuronal marker (Islet-1) were clearly increased (FIG. 3). In conclusion, the method for inducing differentiation of the present invention induced the differentiation of mesenchymal stem cells into neurons and further exhibited the potential for inducing differentiation into motor neurons.

### <1-4> Detection of Neuronal Differentiation Markers by Immunofluorescence Cytochemistry Method

A cover slip (Fisher Scientific, USA) was coated with 20 μg/ml of PDL (Sigma, USA) for a day, which was then re-coated with 10 μg/ml of laminin (Sigma, USA) for three hours, followed by washing with distilled water 5 times. The growing mesenchymal stem cells were detached from the culture vessel using 0.1% trypsin/EDTA, which were seeded on the prepared PDL-laminin coated cover slip and then cultured in DMEM (containing 1000 mg/l glucose, WelGENE, Korea) supplemented with 10% FBS for a day. Non-treated control mesenchymal stem cells or NIM-treated stem cells of Example <1-2> were treated with 4% (v/v) paraformaldehyde at room temperature for 30 minutes, followed by washing with 1× PBS three times. Then, the cells were treated with 0.2% triton X100 for 10 minutes, permeated and washed with 1× PBS three times. To avoid non-specific binding, blocking buffer (1× PBS containing 5% goat serum) was treated thereto for one hour. The primary antibody (Table 2) diluted in the blocking buffer at 4° C. was bound to the mesenchymal stem cells overnight, followed by washing with 1× PBS three times. Then, FITC(fluorescein isothiocyanate)-binding or Cy3-binding-anti-mounse or anti rabbit (Jackson Immunoresearch, USA) secondary antibody diluted in the blocking buffer (1:500) was bound to the stem cells for one hour at room temperature in a dark room. The nuclei were stained with DAPI (Santa Cruz, USA) diluted in PBS (1:2,500) for 10 minutes, washed with 1× PBS three times and then photographed by a confocal microscope (FluoView™ Confocal Microscope, Olympus, Japan), followed by histological analysis.

As a result, the stem cell marker nestin was expressed at Oh but the expression was reduced over the NIM treating times. The expressions of neuronal markers, such as NeuN, MAP-2 and NF-M (neurofilament 150 kDa) were increased over the NIM treating times and their morphology was more like neuron-like cells, suggesting that the stem cells were being differentiated into neurons (FIG. 4).

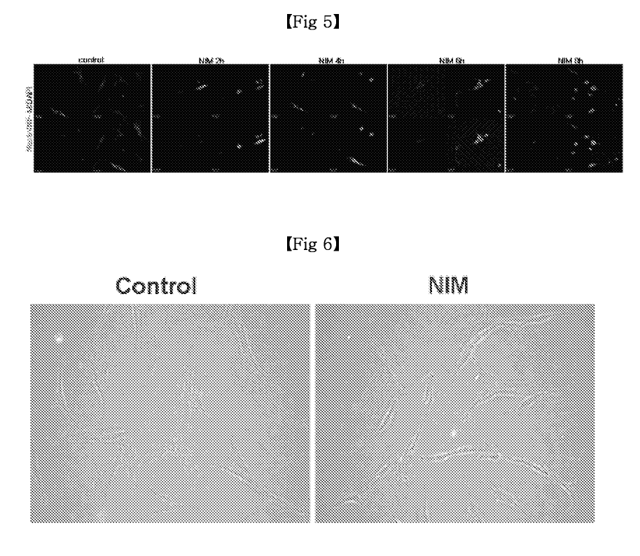

The expressions of neuronal marker proteins were detected over the NIM treating times (2, 4, 6 and 8 hours) by immunofluorescence. As a result, NF-M expression was increased in NIM 6 group (cells were treated with NIM for 6 hours), whereas nestin expression was reduced therein (FIG. 5).

TABLE 2
Table 2: Dilution ratios of primary antibodies

| Name | Dilution ratio | Host |
|---|---|---|
| Nestin | 1:300 | Mouse |
| NF-M | 1:400 | Rabbit |
| NeuN | 1:500 | Mouse |
| MAP2 | 1:200 | Mouse |

### COMPARATIVE EXAMPLE 1

### Recurrence of Differentiation of Mesenchymal Stem Cells into Neurons by the Method of Zhao et al

<1-1> Induction of Differentiation of hMSC into Neurons

The present inventors induced differentiation of hMSC into neurons according to the method of Zhao et al (Zhao et al., Exp Neurol, 190, 396-406, 2004). The mesenchymal stem cells prepared in Experimental Example <1-1> were cultured in a general medium (DMEM/F-12 containing 10% FBS and penicillin-streptomycin) and then transferred into another medium supplemented with 1 mM β-mercaptoethanol (Sigma, USA) and cultured for 24 hours to induce neuronal differentiation. The medium was replaced with another general medium supplemented with 2 mM β-mercaptoethanol and the stem cells were further cultured for 3 hours, leading to the pre-induction. The medium was replaced again with a neuronal induction medium (NIM) prepared by adding 2% DMSO (Sigma, USA) and 200 μM butylated hydroxyanisole (Sigma, USA) to DMEM supplemented with N2 supplements (Gibco, USA) instead of 10% FBS, followed by further culture for one˜two more hours. The changes of morphology were observed under a microscope.

As a result, the differentiation of mesenchymal stem cells into neurons was not confirmed by morphological changes, suggesting that the recurrence of differentiation was not successful (FIG. 6).

### <1-2> Detection of Neuronal Differentiation Markers by RT-PCR

The method to extract RNA from non-treated control mesenchymal stem cells and NIM-treated mesenchymal stem cells of Comparative Example <1-1> and the method to detect a band using electrophoresis and transilluminator after reverse transcription and PCR were the same as described in Experimental Example <1-3>.

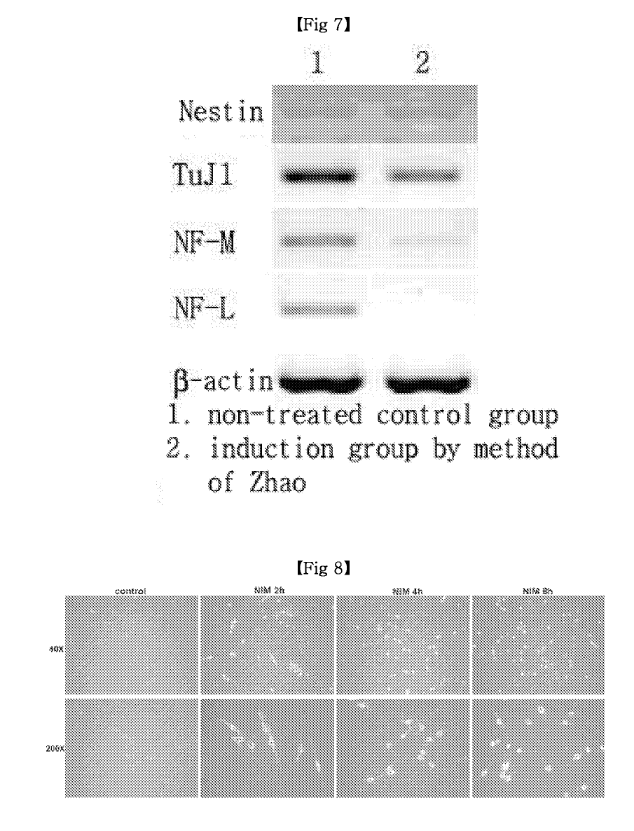

As a result, the level of neuronal marker was not increased by the differentiation of mesenchymal stem cells into neurons according to the procedure of Comparative Example <1-1> (FIG. 7). This result indicates that differentiation induction by the method of Zhao et al was not as efficient as the method of the present invention.

### COMPARATIVE EXAMPLE 2

### Recurrence of Differentiation of Mesenchymal Stem Cells into Neurons by the Combination of the Method of Zhao et al and the Method of Bertani et al

<2-1> Induction of Differentiation of hMSC into Neurons

The present inventors induced differentiation of hMSC into neurons by the combination of the method of Zhao et al (Zhao et al., Exp Neurol, 190, 396-406, 2004) and the method of Bertani et al (Bertani N et al., J Cell Sci, 118, 3925-3936, 2005). The mesenchymal stem cells prepared in Experimental Example <1-1> were cultured in a general medium (DMEM containing 10% FBS and penicillin-streptomycin) and then transferred into another general medium supplemented with 1 mM β-mercaptoethanol (Sigma, USA), which were cultured for 24 hours to induce neuronal differentiation. The medium was replaced with another general medium supplemented with 2 mM β-mercaptoethanol and the stem cells were further cultured for 3 hours, leading to the pre-induction. The medium was replaced again with a neuronal induction medium (NIM) prepared by adding 2% DMSO (Sigma, USA) and 200 μM butylated hydroxyanisole (Sigma, USA), 10 μM forskolin (Sigma, USA), 2 mM valproic acid (Sigma, USA) and 10 mM potassium chloride (Sigma, USA) to DMEM supplemented with N2 supplements (Gibco, USA) instead of 10% FBS, followed by further culture for a required time (0, 3, 6, 9 hours and 1 day).

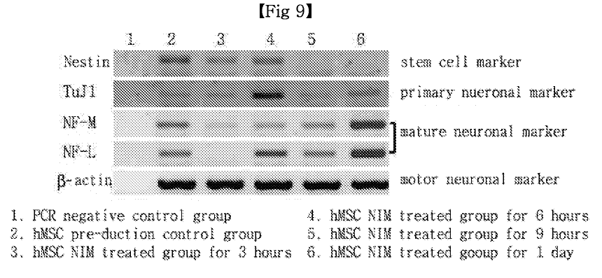

As a result, the differentiation of mesenchymal stem cells into neurons and recurrence of the differentiation was improved, compared when the method of Zhao et al was used alone (FIG. 9). And, morphological analysis also confirmed the differentiation of mesenchymal stem cells into neurons (FIG. 8).

### <2-2> Detection of Neuronal Differentiation Markers by RT-PCR

The method to extract RNA from non-treated control mesenchymal stem cells and NIM-treatedmesenchymal stem cells of Comparative Example <2-1> and the method to detect a band using electrophoresis and transilluminator after reverse transcription and PCR were the same as described in Experimental Example <1-3>.

As a result, the expression of the neuronal marker was detected during the differentiation of mesenchymal stem cells into neurons according to the method of Comparative Example <2-1>, suggesting that this method is more efficient in inducing differentiation of mesenchymal stem cells into neurons than the method of Zhao et al (FIG. 9).

### INDUSTRIAL APPLICABILITY

As explained hereinbefore, the method of the present invention composed of double pre-inductions and using neuronal induction media (NIM) supplemented with butylated hydroxyanisole, forskolin and VPA can provide neurons or motor neurons for the cell therapy of neurodegenerative diseases by inducing differentiation of mesenchymal stem cells into neurons or motor neurons.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for inducing differentiation of mesenchymal stem cells into neurons, comprising the following steps:
1) Performing pre-induction of mesenchymal stem cells twice; and
2) Inducing differentiation of the pre-differentiated mesenchymal stem cells of step 1) in a neuronal induction medium containing butylated hydroxyansiole (BHA), forskolin and valproic acid (VPA) for 2˜8 hours.

2. The method for inducing differentiation of mesenchymal stem cells into neurons according to claim 1, wherein the content of β-mercaptoethanol added for the second pre-induction is 1.5˜2 fold increased from the content added for the first pre-induction.

3. The method for inducing differentiation of mesenchymal stem cells into neurons according to claim 1, wherein the second pre-induction time is reduced to ¼˜⅛ of the first pre-induction time.

4. The method for inducing differentiation of mesenchymal stem cells into neurons according to claim 1, wherein the second pre-induction time is reduced to ⅛ of the first pre-induction time.

5. The method for inducing differentiation of mesenchymal stem cells into neurons according to claim 1, wherein the neuronal induction medium contains 100˜200 μM BHA, 9˜11 μM forskolin and 1.5˜2.5 μM VPA.

6. The method for inducing differentiation of mesenchymal stem cells into neurons according to claim 1, wherein the induction was performed with the neuronal induction medium for 2˜8 hours.

7. A neuronal induction medium containing butylated hydroxyanisole (BHA), forskolin and valproic acid (VPA) to induce differentiation of pre-induced mesenchymal stem cells into neurons.

8. The neuronal induction medium according to claim 7, which contains 100˜200 μM BHA, 9˜11 μM forskolin and 1.5˜2.5 μM VPA.
